(19) United States
(12) Patent Application Publication
(10) Pub. No.: US 2022/0016154 A1
(43) Pub. Date: Jan. 20, 2022

(54) TREATMENT OF NEURONAL MOSAICISM IN DEMENTIA

(71) Applicant: Crownview Medical Group, Inc., Coronado, CA (US)

(72) Inventor: Mark Melden, Coronado, CA (US)

(73) Assignee: Crownview Medical Group, Inc., Coronado, CA (US)

(21) Appl. No.: 17/378,626

(22) Filed: Jul. 16, 2021

Related U.S. Application Data
(60) Provisional application No. 62/705,827, filed on Jul. 17, 2020.

(51) Int. Cl. A61K 31/7072

(52) U.S. Cl. CPC A61K 31/7072

(57) ABSTRACT

A method for the use of at least one reverse transcriptase inhibitor, that when administered in a safe and effective amount, ameliorate at least one of the formation, maintenance, or expression of excessive neuronal mosaicism, and therefore decrease or block neurogenic mechanisms underlying or associated with dementia.

### CROSS-REFERENCE TO RELATED APPLICATION

The present application claims the benefit of U.S. Provisional Patent Application No. 62/705,827 filed on Jul. 17, 2020, and titled, “TREATMENT OF NEURONAL MOSAICISM IN DEMENTIA”, the disclosure of which is expressly incorporated by reference in its entirety.

### BACKGROUND

### Field

Certain aspects of the present disclosure generally relate to treatment of dementia, and more particularly to systems and methods for the relief or prevention of dementia symptoms using Non-Nucleoside Reverse Transcriptase Inhibitors (NNRTI), Protease Inhibitors (PI), Nucleoside/Nucleotide Reverse Transcriptase Inhibitors (NRTIs), or some combination thereof.

### Background

Most types of dementia cannot be cured. The symptoms of dementia may in some cases be managed with medications and therapies. Cholinesterase inhibitors, such as donepezil, rivastigmine, and galantamine, may be used to temporarily improve dementia symptoms. The mechanism of action for cholinesterase inhibitors is thought to involve the boosting of levels of a chemical messenger, acetylcholine, involved in memory and judgment. Another medication, memantine, is thought to work by regulating the activity of glutamate, which is another chemical messenger in the brain.

Therapies for dementia include occupational therapy, reducing clutter, and simplifying the tasks that a person with dementia must perform on a daily basis. These therapeutic interventions may help reduce confusion in people with dementia.

Aspects of the present disclosure provide for the treatment of dementia using medications that have the potential for longer-term amelioration of dementia symptoms and their progression.

### SUMMARY OF THE INVENTION

Aspects of the present disclosure provide a method. The method generally includes administering a therapeutically effective amount of reverse transcriptase inhibitor, whereby the reverse transcriptase inhibitor interrupts a neurogenic pathway associated with neuronal mosaicism.

In some embodiments, the reverse transcriptase inhibitor is selected from the group consisting of NRTIs, NNRTIs, and PIs, which may be administered alone or in combination. In some embodiments, the reverse transcriptase inhibitor is Zidovudine (Retrovir).

In some embodiments, the method further includes administering a mental status exam and determining a score of the administered mental status exam, wherein the reverse transcriptase inhibitor is selected based at least in part on the determined score. In some embodiments, a therapeutically effective amount of a reverse transcriptase inhibitor is determined based at least in part on the determined score.

### BRIEF DESCRIPTION OF THE DRAWINGS

FIG. 1 illustrates Reverse Transcription and the action of NNRTIs and NRTIs in a somatic cell of the brain.

FIG. 2 illustrates DNA integration and the action of an Integrase Inhibitor in a nucleus of a somatic cell of the brain.

FIG. 3 illustrates DNA replication in a diseased cell and the action of Protease Inhibitors.

FIG. 4 illustrates normal and abnormal cleavage of amyloid precursor protein (APP).

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. However, it will be apparent to those skilled in the art that these concepts may be practiced without these specific details. In some instances, well-known structures and components are shown in block diagram form in order to avoid obscuring such concepts.

Based on the teachings, one skilled in the art should appreciate that the scope of the disclosure is intended to cover any aspect of the disclosure, whether implemented independently of or combined with any other aspect of the disclosure. For example, an apparatus may be implemented, or a method may be practiced using any number of the aspects set forth. In addition, the scope of the disclosure is intended to cover such an apparatus or method practiced using other structure, functionality, or structure and functionality in addition to or other than the various aspects of the disclosure set forth. It should be understood that any aspect of the disclosure disclosed may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the disclosure. Although some benefits and advantages of the preferred aspects are mentioned, the scope of the disclosure is not intended to be limited to particular benefits, uses or objectives. Rather, aspects of the disclosure are intended to be broadly applicable to different technologies and system configurations, some of which are illustrated by way of example in the figures and in the following description of the preferred aspects. The detailed description and drawings are merely illustrative of the disclosure rather than limiting, the scope of the disclosure being defined by the appended claims and equivalents thereof.

It must be noted that as used herein and in the appended claims, the singular forms “a”, “an”, and “the” include plural referents unless the context clearly dictates otherwise. Thus, for example, reference to “an object” includes a plurality of objects.

### Neuronal Mosaicism

A traditional view of genomics posits that all cells of an individual have identical genomes, and that cellular diversity results from differences in transcription in different cells. A prominent exception to this view emerged in the 1960s. Changes in a DNA sequence which occur after birth, and which may include gene recombination, produce new, combinatorial coding sequences. The resultant diversity of genomes in different cells of an organism based on such post-birth DNA changes is said to form a complex “mosaic” of genetic material.

A central mechanism of neuronal mosaicism is reverse transcription, a process that culminates in an insertion of genetic material into the genome of a cell. Transcription describes the process by which an RNA molecule is read from a sequence of DNA. In reverse transcription, rather than using DNA to produce new RNA, RNA may be used to produce new DNA. When the DNA that is inserted back into the genome differs from what is already present, the cell's genome has changed. Reverse transcription is found naturally in the human body and is thought to play a role in the body's ability to respond to infection.

Human Immunodeficiency Virus (HIV) uses reverse transcriptase to copy its genome into the genome of infected cells. Reverse transcriptase inhibitors were developed to block this process and thereby halt the progression of HIV-AIDS.

DNA sequences have also been found to vary from neuron to neuron in the brain. In particular, it has been observed that genetic recombination of the gene that encodes the amyloid precursor protein (APP) occurs in neurons in the human brain. Furthermore, neurons show greater APP gene diversity in individuals with sporadic Alzheimer's disease compared with healthy individuals. These observations indicate that normal and diseased brains may be considered a “mosaic” of distinct genomes, and furthermore, suggest that genetic recombination processes are heighted in sporadic Alzheimer's disease.

Dementia is a syndrome characterized by a group of symptoms affecting mental cognitive tasks such as memory and reasoning. Alzheimer's Disease (AD) is the most common condition associated with Dementia. Alzheimer's Disease (AD), first described by Alois Alzheimer in 1907, is a progressive neurological disorder. Early symptoms tend to include short-term memory loss. Later stages include disorientation, impairment of judgment, reasoning, attention and speech and, ultimately, dementia. The societal cost for managing AD currently exceeds $100 billion annually, primarily due to the extensive custodial care required for AD patients. Despite continuous efforts aimed at understanding the physiopathology of AD, there is currently no treatment that significantly retards the progression of the disease.

While the causal link between APP and dementia remains unclear, various observations suggest that APP-encoding gene variants may contribute to the formation of Alzheimer's disease. Thus, the variety of APP encoding genes may be heightened in Alzheimer's disease. Amyloid-Beta, which is associated with APP, can drive persistent neuroinflammation by binding to microglia and by causing the production of inflammatory cytokines. APP also leads to neurofibrillary tangles, a commonly observed biomarker of Alzheimer's disease.

In some embodiments of certain aspects of the present disclosure, by blocking conversion of APP RNA into new proteins at one or more locations in the reverse transcription pathway, fewer copies of abnormal APP may be produced. This, in turn, may cause a reduction in neuroinflammation. Relief from neuroinflammation may then account for an observed amelioration of dementia symptoms, as disclosed herein.

### Imbalance of Reverse Transcriptase and Polymerase

Based on the insight that the brain works as a mosaic, and the further observation that the variation of a brain's mosaic is increased in certain forms of dementia, certain aspects of this disclosure are directed to the treatment of dementia with medications that target the biochemical processes that are implicated in the formation, maintenance, and/or expression of neuronal mosaicism.

The process by which the information in a strand of DNA is copied into a new molecule of messenger RNA (mRNA) is commonly referred to as Transcription. Whereas DNA may be considered a secure and stable store of genetic material in the nuclei of cells, much like a reference library, mRNA may be thought of as a hand-written copy from a reference book. mRNA is not used for long-term storage and it can freely exit the nucleus. When transcription is error-free, the mRNA carries the same information as the corresponding strand of DNA.

RNA polymerase is an enzyme that catalyzes the formation of RNA from a DNA template during transcription. Transcription additionally involves a number of accessory proteins known as transcription factors. Transcription factors can bind to specific DNA sequences and then recruit RNA polymerase to an appropriate transcription site.

Reverse Transcriptase (RT) causes RNA strands to create new DNA strands which may then be incorporated into the DNA of the molecule. The new DNA strands may also remain outside of the nucleus. DNA strands that remain outside of the nucleus may be referred to as viral DNA, since they may be considered analogous to exo-nucleic DNA strands that have entered the cell as part of a virus, as in HIV.

DNA strands produced by Reverse Transcriptase may contain errors and therefore may be inexact copies of the original nucleic DNA strands. These RT-produced DNA strands may be used in subsequent protein synthesis, in some cases, before the DNA strands are incorporated back into the nucleic DNA of the cell. The proteins produced by this pathway may be slightly different than what would be produced from protein synthesis based directly from RNA polymerase transcription of the original DNA in the nucleus.

According to certain aspects the present disclosure, when the brain is in a state of mosaic dysfunction, individual cells may rely more heavily on RT as a promoter of certain types of protein synthesis. RT may be so active in certain brain cells that the presence of RT-based DNA snippets that encode certain proteins may overwhelm what may be produced by the RNA polymerase pathway. The protein synthesis due to RT versus RNA polymerase may be pathologically out of balance.

Accordingly, certain aspects of the present disclosure are directed to blocking or reducing the efficacy of Reverse Transcription. This may restore a more natural balance such that RNA polymerase takes on a relatively more substantial role in protein synthesis in the diseased brain.

### Reverse Transcriptase Inhibitors to Treat Dementia

According to certain aspects of the present disclosure, dementia patients, including dementia patients who are not diagnosed as having AD, may be administered Reverse Transcriptase Inhibitors (RTIs). RTIs may target protein synthesis pathways in the diseased brain in which reverse transcriptase and associated downstream processes assume an enhanced role, relative to RNA polymerase, in the synthesis of proteins in neurons in the brain. RTIs may include Nucleoside/Nucleotide reverse transcriptase inhibitors (NRTIs), Non-nucleoside reverse transcriptase inhibitors (NNRTIs), and Protease Inhibitors (PIs), which may be administered alone or in combination.

NRTI is a generic term covering a family of medications, comprising: Abacavir (ABC, Ziagen), Emtricitabine (FTC, Emtriva), Lamivudine (3TC, Epivir), Tenofovir Alafenamide (TAF, Vamlidy), Tenofir Disporoxil Fumarate (TDF, Viread), and Zidovudine (ZDV, Retrovir). NRTIs work by targeting the action of reverse transcriptase.

NNRTI is a generic term covering a family of medications, comprising Delavirdine (DLV, Rescripor), Doravirine (DOR, Pifeltro), Efavirenz (EFV, Sustiva), Etravirine (ETR, Intelence), Nevirapine (NVP, Viramune), and Rilpivirine (RPV, Edurant). NNRTI block reverse transcription.

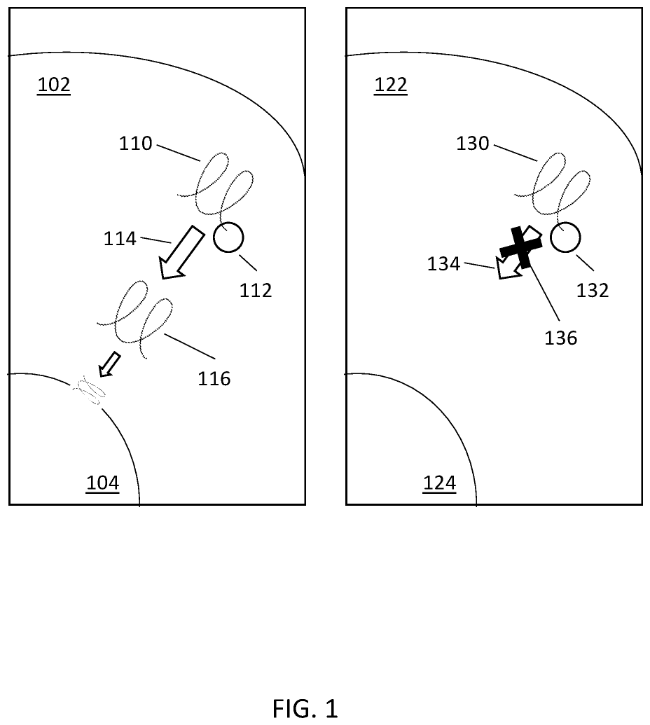

FIG. 1 illustrates how NNRTIs and NRTIs may block reverse transcription and thus ameliorate dementia symptoms and or disease progression, in accordance with certain aspects of the present disclosure. The soma cell body 102 may contain APP RNA 110. A reverse transcriptase 112 may cause the transcription 114 of APP DNA 116. The APP DNA 116 may then make its way into the soma cell nucleus 104. In the right panel, the soma cell body 122 is shown as modulated by an NNRTI and/or NRTI. Again, there is APP RNA 130 and a reverse transcriptase 132. Here however, the transcription 134 is blocked 136 by the action of an NNRTI, an NRTI, or some combination thereof.

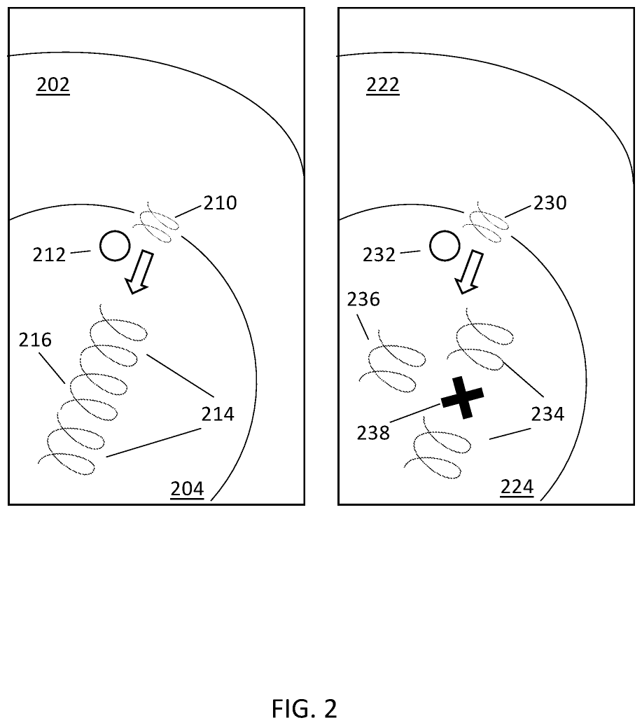

FIG. 2 illustrates Integration that may follow the insertion of APP DNA. In FIG. 2, APP DNA 210 travels from the soma cell body 202 to the soma cell nucleus 204. Inside the nucleus, an integrase 212 may cause the APP DNA 210 that entered the nucleus to become integrated within existing DNA 214 at site 216. According to certain aspects of the present disclosure, integration of APP DNA may be blocked at this stage. As shown in the right panel of FIG. 2, the APP DNA 230 may travel from the soma cell body 202 to the soma cell nucleus 224. An integrase 232 may then be blocked 238 or prevented from integrating the APP DNA 236 into the existing DNA 234.

PI is a generic term covering a family of medications, comprising: Atazanavir (ATV, Reyataz), Darunavir (DRV, Prezista), Fosamprenavir (FPV, Lexiva), Indinavir (IDV, Crixivan), Lopinavir+ritonavir, (LPV/r, Kaletra), Nelfinavir (NFV, Viracept), Ritonavir (RTV, Norvir), Saquinavir (SQV, Invirase, Fortovase), and Tipranavir (TPV, Aptivus). PIs act downstream of NRTIs and NNRTIs to hyrdrolyze peptide bonds of proteins.

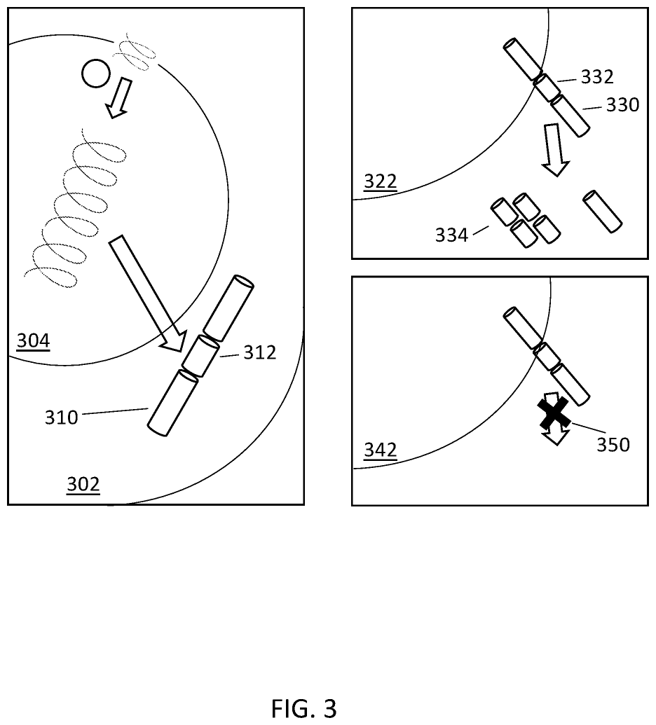

FIG. 3 illustrates how PIs may block protein cleavage, and thus ameliorate dementia symptoms and or disease progression, in accordance with certain aspects of the present disclosure. In the left panel of FIG. 3, the soma cell nucleus 304 is shown having DNA with integrated APP DNA that entered the soma cell nucleus 304 from a reverse transcription pathway. The APP DNA is read out into a protein that travels to the soma cell body 302. In this illustration, the protein consists of three segments, including segment 312 and segment 310. In the top right panel, this protein segment had emerged from the cell membrane surrounding the soma cell body 322. Two of the protein segments are in the extracellular space, including segment 330 and segment 332. A protease may then cause these segments to be cleaved from the cell membrane, and further cleaved from each other. Over multiple similar event, segments such as the middle segment 332 may build up 334.

In the bottom right panel of FIG. 3, the action of a PI is illustrated. Here, the protein still protrudes from the membrane surrounding the soma cell body 342. A PI, however, blocks 350 the action of the protease. This prevents the accumulation of segments that is thought to lead to the formation of plaques.

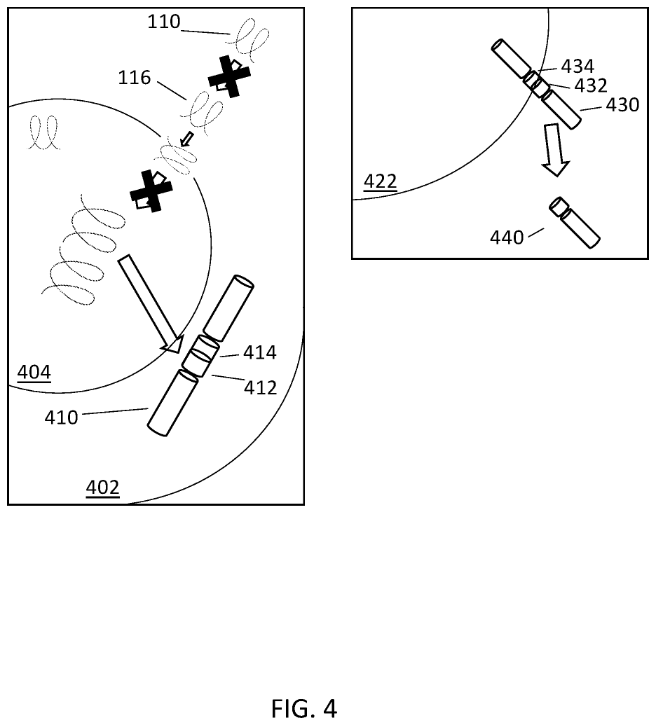

FIG. 4 illustrates the transcription of APP from unmodified DNA. In the soma cell body, there may be APP RNA 110. This APP RNA may be blocked from being transcribed into APP DNA 116. In addition, or alternatively, APP DNA 116 that makes its way into the soma cell nucleus 404 may be blocked from integrating into the existing DNA of the cell. The un-modified DNA may then produce a normal protein. Here, there is again an end segment 410, and the middle protein segment is broken into two piece, a first segment 412 and a second segment 414. When the protein reaches the cell membrane, one of the end segment 430 and the first segment 432 are cleaved such that they remain together 440. The second segment 434 is cleaved apart. In accordance with certain aspects of the present disclosure, the pathways illustrated in FIG. 4 become more prominent in the brain, so that on balance the accumulation of plaque materials are reduced.

Support for the potential of this approach is bolstered by observations from the community of patients having HIV. It has been noted that AIDS patients have been less likely to develop dementia symptoms than would be expected for age-controlled groups. This observation has been limited to patients who took RTI medications. Furthermore, the progression of HIV-associated dementia was observed to be slowed by administration of Zidovudine, an antiretroviral therapy.

Researchers have previously proposed (U.S. Pat. No. 10,100,307, College de France) the use of reverse-transcriptase inhibitors in the prevention and treatment of a wide range of degenerative diseases, including Parkinson's disease, Alzheimer's disease, Huntington's disease, amyotrophic lateral sclerosis (ALS), and degenerative diseases affecting eyesight or hearing, especially glaucoma, based on the observation that RTIs reduce the formation of double-stranded DNA breaks (DSBs) under conditions of oxidative stress in mice. The present disclosure, in contrast, is directed to treatment of dementia syndrome observed in humans, such that RTI treatment may be prescribed in a safe, effective, and targeted manner based on psychiatric evaluations.

Furthermore, whereas previously researchers have relied upon screening of patients based on the presence of unwanted accumulation of amyloid beta protein (PCT Patent Application US2018/030520, Sanford Burnham Prebys Medical Discovery Institute), certain aspects of the present disclosure provide for screening based on clinical evaluation of dementia symptoms. As such, according to certain aspects, a patient may be beneficially treated with RTIs prior to the development of detectable levels of amyloid plaques.

The present invention provides a safe and effective method for the treatment of dementias that have a neurogenic component to a response to RTIs. The present invention includes the use of RTIs which inhibit neural mosaic formation, expression, and maintenance related to dementia phenotypes. In some embodiments, an effective treatment regimen may include NNRTIs, either alone or in combination with NRTIs and PIs. In some embodiments, an effective treatment regimen may include a PI taken alone, an NRTI taken alone, or some combination thereof, as described below. According to certain aspects, the therapeutic effect of RTIs may involve (but may not be limited to) an inhibition of formation, expression, and/or maintenance of a neural mosaic.

Certain aspects of the present disclosure alleviate dementia symptoms. As disclosed herein, a dementia patient undergoing a prescribed course of RTIs may experience improvements in an ability to remember one's own home address, to articulate how to navigate to one's own home address, to recognize people who the patient only recently met, and the like.

Certain aspects of the present disclosure provide dosages that are effective in the therapeutic treatment of different types of dementia. Dosage may depend on severity of dementia, as evidenced by SLUMS and/or MMSNE scores. In an exemplary embodiment, Retrovir may be prescribed. A safe and effective dosage could be as high as 1000 mg per day but a more useful range of 200 to 600 mg per day, divided into equal amounts and administered twice or three times daily, alone or in combination with other compounds, may be more suitable. However, higher or lower dosages may be necessary.

In some embodiments, it may be advantageous to pursue a regimen of primarily NNRTI-based treatment. One approach may be referred to as an NRTI-sparing regimen. In this approach, NRTIs may be excluded from the treatment regimen to avoid certain side effects that are associated with NRTIs. In some embodiments, NRTIs may be avoided initially to prevent the development of drug resistance to NRTIs, so that a person with dementia may still beneficially use NRTIs in future treatment regimens.

In some embodiments, it may be advantageous to pursue a regimen that emphasizes PI-based treatment. PIs, by blocking a protein that diseased cells need to put together new proteins, may effectively shift production of such proteins to healthier cells, which may be less error-prone and/or less pathogenic.

In some embodiments, it may be advantageous to pursue a regimen of primarily NRTI-based treatment. As explained above, RNA polymerase may provide for higher fidelity protein synthesis than reverse transcriptase. Blocking reverse transcriptase, therefore, may help preserve brain function and may decrease the risk of developing plaques and/or neurofibrillary tangles.

By way of analogy to treatment approaches for HIV, a regimen for a particular patient exhibiting dementia symptoms may be guided by factors such as dementia severity, toxicity, pill burden, dosing frequency, drug-drug interaction potential, resistance test results, comorbid conditions, access, and cost. NNRTI-based regimens may be options for some patients, although these drugs, especially EFV and RPV, have low barriers to resistance. EFV has a long track record of widespread use, and is considered safe in persons of childbearing potential. When choosing an RTI regimen, people with dementia, their family members, caretakers, and their health care providers should consider the following non-limiting factors: other diseases or conditions that the person with dementia may have, such as heart disease; possible side effects of the medications; convenience of the regimen (for example, a regimen that includes two or more RTIs may be more convenient to follow if the medications are combined into one pill). Issues that can make it difficult to follow an RTI regimen should be considered. For example, a lack of health insurance or an inability to pay for RTI medicines may make it challenging to take RTI medicines consistently every day.

### Selecting Patients and Measuring Outcomes

Dementia symptom severity may be measured and tracked with reference to standardized clinical tests, such as the Standardized Mini-Mental State Examination (SMMSE) or the Saint Louis University Mental Status Examination (SLUMS). The SSMSE exam includes questions such as, “What city are we in?” If the test is administered in the patient's home, the test includes the question, “What is the street address of this house?” Other tests of the SSMSE exam include asking the patient to spell the word “WORLD” backwards; or to show the patient a pencil and ask the patient, “What is this called?”. A SLUMS exam includes questions such as: “What day of the week is it?”, “What is the year?”, and further includes data manipulation tests, such as, “You have $100 and you go to the store to buy a dozen apples for $3 and a tricycle for $20. How much did you spend? How much do you have left?” For a dementia treatment regimen, contemplated measures of efficacy include noting improvements, decline, or stability in an individual's scores in such tests. Alternatively, or in addition, measures of efficacy may be more narrowly tailored to specific cognitive impairments of the individual undergoing treatment.

In some embodiments, RTIs may be prescribed for a patient in advanced stages of dementia, which may be characterized, for example, by SMMSE scores that are consistently below 14.

In accordance with certain aspects of the present disclosure, a patient with symptoms of early stage dementia was administered the SMMSE to establish a baseline of her mental status. She scored 27 out of a 30. Seven years later, after her dementia has advanced substantially, she was scoring between 2 and 4 on the same test. This patient was administered a regimen of ZDV (Retrovir). After 8 weeks of treatment, the patient appeared more alert and aware of her surroundings and demonstrated specific improvements in her ability to remember and communicate about where she lived. For the first time in two years she was able to recite her home address. Upon further questioning, she was able to provide directions from the facility in which she was being examined to her home address. Still, the patient's SMMSE scores were not consistently improved.

For some patients, results of clinical mental status exams may be unaffected, and yet a patient's caregiver may advocate for continued treatment due to observed improvements in one or more aspects of the patient's life, which may not be measured by the mental status exams. For some dementia patients, for example, renewed interest in social engagement or reduced signs of frustration in a social context may indicate an improvement to a patient who is at risk of social isolation. In some embodiments, measurements of efficacy of RTI treatment may incorporate subjective feedback provided by a caregiver associated with the patient.

What is claimed is:

1. A method, comprising:
identifying a patient as having dementia, and
administering a therapeutically effective amount of reverse transcriptase inhibitor, whereby the reverse transcriptase inhibitor interrupts a neurogenic pathway associated with neuronal mosaicism.

2. The method of claim 1, wherein the reverse transcriptase inhibitor is selected from the group consisting of NRTIs, NNRTIs, and PIs.

3. The method of claim 1, wherein the reverse transcriptase inhibitor comprises Zidovudine.

4. The method of claim 3, wherein the dosage is less than about, or equal to 400 mg per day.

5. The method of claim 1, further comprising:
administering a mental status exam; and
determining a score of the administered mental status exam.

6. The method of claim 5, wherein the mental status exam is the Saint Louis University Mental Status Examination.

7. The method of claim 5, wherein the mental status exam is the Standardized Mini-Mental State Examination.

8. The method of claim 5, wherein the reverse transcriptase inhibitor is selected based at least in part on the determined score.

9. The method of claim 5, wherein the therapeutically effective amount is based at least in part on the determined score.

10. The method of claim 1, wherein the patient is not diagnosed as having Alzheimer's Disease.
